(19) United States
(12) Patent Application Publication
(10) Pub. No.: US 2004/0208983 A1
(43) Pub. Date: Oct. 21, 2004

(54) Antimicrobial coatings for ophthalmic devices

(76) Inventors: Gregory A. Hill, Atlantic Beach, FL (US); Frank F. Molock, Orange Park, FL (US); Jozefowicz Marcel, Lamorlaye (FR); Osman Rathore, Jacksonville, FL (US); Jacqueline Jozefonvicz, Lamorlaye (FR); Zohra Fadli, Villetaneuse (FR)

Correspondence Address: Louis M. Heidelberger, Reed Smith LLP, 2500 One Liberty Place, 1650 Market Street, Philadelphia, PA, 19103-7301, US

(21) Appl. No.: 10/414,880

(22) Filed: Apr. 16, 2003

(51) Int. Cl. A61L 2/00; B05D 5/06

(52) U.S. Cl. 427/2.1; 427/162

(57) ABSTRACT

A method of reducing adverse events with contact lenses by preventing microbial growth by attaching a polymer (with a molar ratio of carboxylate groups to sulfonate groups of greater than about 2) to a surface of an ophthalmic device is presented. Additionally, a method of promoting ocular health by attaching a polymer (with a molar ratio of carboxylate groups to sulfonate groups of greater than about 2) to a surface of an ophthalmic device is presented.

### FIELD OF THE INVENTION

The present invention relates to antimicrobial, including antibacterial, coatings for ophthalmic devices, preferably polymer coatings for contact lenses.

### BACKGROUND OF THE INVENTION

Contact lenses have been used commercially to improve vision since the 1950s. Technology has progressed so that contact lenses are now used daily (and removed at night for cleaning) or for up to several days without removal for cleaning. While prolonged use of contact lenses is convenient for the consumer, there are some problems which may arise with prolonged use.

Prolonged use of contact lenses may allow for increased proliferation and accumulation of bacteria or other microbes, such as, but not limited to, Pseudomonas aeruginosa, Acanthamoeba species, Staphylococcus aureus, Escherichia coli, Staphylococcus epidermidis, and Serratia marcesens, on the surfaces of the contact lenses. This accumulation can cause side effects such as contact lens acute red eye. Other adverse effects associated with microbial growth and attachment may include, but are not limited to, contact lens associated red eye, infiltrative keratitis, and microbial keratitis.

There have been varied efforts to reduce this proliferation and accumulation of bacteria and other microbes on the surface of contact lens.

Prior art approaches have used materials such as organic materials, drugs or heavy metals to kill bacteria and other microbes. However, antimicrobial compounds may lead to resistance of the microbe to the drug, and heavy metals may have undesirable side effects (long term effects are unknown).

Inhibition of growth of bacteria and/or other microbes has been attempted in the art, with silver incorporated into contact lenses using silver or a silver zeolite (see European Patent Application EP 1050314 A1), incorporated herewith by reference. However, microbial growth or adhesion to contact lenses remains a troubling problem in the art.

Thus there remains a need for contact lenses which inhibit and/or do not promote bacterial and/or other microbial growth and/or adhesion to the surface of the contact lens. Additionally, there is a need for contact lenses that inhibit adverse responses in the wearer related to the growth of bacteria and/or other microbes.

### SUMMARY OF THE INVENTION

The present invention prevents strong attachment of microbes to the ophthalmic devices thereby allowing the consumer's natural defenses to remove a substantial amount of the microbes from the ocular environment before adverse effects occur. In addition, the proliferation of attached bacteria or other microbes is reduced. The present invention reduces the adhering bacteria or other microbes and reduces their proliferation rate on ophthalmic devices and therefore make the ophthalmic devices safer for humans, especially for contact lens wear.

More specifically, this invention includes a method of preventing microbial attachment on ophthalmic devices, preferably contact lenses, by attaching a polymer to one or more surfaces of an ophthalmic device, wherein said polymer has a molar ratio of greater than about 2 carboxylate groups to about 1 sulfonate groups. Further, this invention includes a method of preventing microbial growth on ophthalmic devices, preferably contact lenses, by attaching a polymer to one or more surfaces of an ophthalmic device wherein said polymer has a molar ratio of great than about 2 carboxylate groups to about 1 sulfonate group) and placing the ophthalmic device on a cornea.

### DESCRIPTION OF THE DRAWINGS

FIG. 1 is a plot of the non-mediated P. aeruginosa adhesion (expressed as the percentages of adhered bacteria per lens) on unfunctionalized and functionalized lenses.

FIG. 2 is a plot of the plasma mediated P. aeruginosa adhesion (expressed as the percentages of adhered bacteria per lens) on unfunctionalized and functionalized lenses.

FIG. 3 is a plot of the plasma mediated P. aeruginosa adhesion (expressed as the percentages of adhered bacteria per lens) on functionalized lenses as a function of MA/SS ratio.

FIG. 4 is a plot of reduction of plasma mediated P. aeruginosa adhesion on functionalized lenses compared to unfunctionalized lenses.

FIG. 5 is a plot of the non-mediated S. aureus adhesion (expressed as the percentages of adhered bacteria per lens) on unfunctionalized and functionalized lenses characterized by the MA/SS ratio.

FIG. 6 is a plot of the plasma mediated S. aureus adhesion on (expressed as the percentages of adhered bacteria per lens) on functionalized lenses compared to unfunctionalized lenses.

FIG. 7 is a plot of S. aureus adhesion (expressed as the percentages of adhered bacteria per lens) on functionalized lenses as a function of MA/SS ratio.

FIG. 8 is a plot of reduction of plasma mediated S. aureus adhesion on functionalized lenses compared to unfunctionalized lenses.

FIG. 9 is a plot of the bacteriophobic effect of lenses coated with random biospecific acrylic tercopolymers with ratio MA/SS of 3.2 (“J&J lenses”) with either P. aeruginosa or S. aureus.

FIG. 10 is a plot of tear-like-mediated bacterial adhesion of P. aeruginosa and S. aureus on functionalized lenses at various MA/SS ratios.

FIG. 11 is a plot of bacterial proliferation of P. aeruginosa on the lenses in the presence of synthetic media or synthetic tear fluid over time.

FIG. 12 is a plot of bacterial proliferation of S. aureus on the lenses in the presence of synthetic media or synthetic tear fluid over time.

### DETAILED DESCRIPTION OF THE PREFERRED EMBODIMENTS

“Functionalized,” as used herein, means the ophthalmic device was coated with at least one random biospecific polymer. A random biospecific polymer has substituents with suitable chemical groups or random copolymerizations of suitable monomers, which contain arrangements of chemical functions which mimic natural biospecific sites. A good background article is Jozefowicz and Jozefonvicz, Randomness and Biospecificity: random copolymers are capable of biospecific molecular recognition in living systems, Biomaterials, 18 (1997) 1633-1644 (incorporated herein by reference).

“Mediated,” as used herein, means at least one ophthalmic device surface has interacted with at least one naturally occurring protein(s) in plasma or tear fluid or tear-like fluid which resulted in a change in the bacterial or other microbial adhesion and proliferation on the ophthalmic device.

Bacteria and other microbes colonize a surface by either a mediated or a non-mediated mechanism. The non-mediated attachment is weak and the microbes are easily removed, generally do not proliferate and are considered a less serious problem than in a mediated attachment. The mediated mechanism utilizes adhesive proteins such as, but not limited to fibronectin, to form a strong attachment to a surface. Adhesive proteins generally have several binding sites for various molecules. The nature (both density and type of charge) of the surface charge may control which protein binding sites interact with the surface and thereby may control the sites available for bacterial or microbial adherence. If the binding sites required for mediated bacterial or other microbial adhesion and proliferation are used to bind the protein to the surface then bacterial or other microbial adhesion and proliferation can be reduced. Binding for the polymer includes but is not limited to chemical bonds, or entanglements (interpenetrating network).

In the present invention, a polymer comprises neutral groups and ionic groups. The ionic groups include but are not limited to carboxylate groups and sulfonate groups, which are attached to the surface of an ophthalmic device, preferably a silicone hydrogel contact lens.

“Preventing microbial attachment,” as used herein, means reducing the amount of microbes which attach to the ophthalmic device, preferably by at least 50%, more preferably about 90% (as compared to microbes attached to ophthalmic devices which do not contain the neutral and ionic groups disclosed herein) and/or inhibiting the ability of microbes to attach to the ophthalmic device.

“Preventing microbial growth,” as used herein, means reducing the growth rate at which microbes grow on an ophthalmic device, preferably by at least 50% (as compared to microbes growing on the surfaces of the ophthalmic device which do not contain the neutral and ionic groups disclosed herein) and/or inhibiting the ability of microbes to attach to the ophthalmic device, and/or killing microbes on the surface of the ophthalmic devices or in an area surrounding the ophthalmic device.

Bacteriophobic properties of an ophthalmic device as used herein means that the device is able to prevent microbial attachment on the device.

Bacteriostatic properties of an ophthalmic device as used herein means that the device is able to prevent microbial growth on the device.

“Ophthalmic devices,” as used herein, mean contact lenses that reside in or on the eye, such as soft contact lenses, hard contact lenses, overlay lenses, ocular lenses and ophthalmic lenses. The contact lenses preferably comprise one or more of the following: poly(methyl)methacrylate polymer, poly(hydroxyethyl)methacrylate polymer, polyacrylic acid polymer, silicone acrylate polymer, fluoroacrylate polymer, fluoroether polymer, polyacetylene polymer, polyimide polymer, hydrogels, silicone materials, acrylic materials, fluorocarbon materials, mixtures and copolymers of any of the foregoing, etafilcon A, genfilcon A, galyfilcon A, lenefilcon A, polymacon, acquafilcon A, balafilcoh A, lotrafilcon A, lotrafilcon B, and silicone hydrogels. The lenses may alternatively comprise or consist of random biospecific polymers.

“Microbes,” as used herein, mean bacteria and other microbes, including but nor limited to, microbes found in or on the eye or tear fluid, particularly Pseudomonas aeruginosa, Acanthamoeba species, Staphylococcus aureus, Escherichia coli, Staphylococcus epidermidis, Serratia marcesens and combinations thereof.

A “synthetic tear fluid,” as used herein, means any fluid that has protein composition and ionic strength properties similar to human tears, including but not limited to a solution of 5% blood plasma supplemented with Lysozyme at 4.5 g/L and Lactoferrin at 1.7 g/L.

“Polymer,” as used herein, means a polymer having one or more carboxylate groups and one or more sulfonate groups, such as methacrylic acid copolymers, methyl methacrylate copolymers, sodium styrenesulfonate copolymers, methyl methacrylate-methacrylic acid-sodium styrene sulfonate random copolymers or combinations thereof. The molar ratio of carboxylate groups to sulfonate groups is preferably greater than about 2, more preferably about 2 to about 4. U.S. Pat. Nos. 6,160,056; 6,218,492; 6,248,811; 6,365,692; and 6,417,000, and U.S. Patent Application Publication No. US2002/0068804 A1, all of which are herein incorporated by reference, teach polymers and the methods of making said polymers, which may be effective in the present invention. Polymers may be prepared by free radical polymerization, condensation polymerization and other methods known to those skilled in the art.

One embodiment of a polymer is a water-insoluble polymer, containing carboxylate and sulfonate groups, obtained by free radical copolymerization of one or more aliphatically unsaturated monomers containing carboxylate groups, or the correspondingly functionalized derivatives of the monomers, as a first component with one or more aliphatically unsaturated monomers containing sulfonate groups, or the correspondingly functionalized derivatives of the monomers, as a second component and a third component which comprises an aliphatically unsaturated monomer, the correspondingly functionalized derivatives being converted into carboxylate and sulfonate groups after the copolymerization.

Another embodiment of a polymer is a water-insoluble polymer obtainable by free-radical polymerization of (a) at least one monomer of the general formula R-Aₐ, in which R is an aliphatically unsaturated organic radical with the valence “a”, A is a carboxyl group, carboxylate group, sulfuric acid group, sulfonic acid group, phosphoric acid group, phosphonic acid group, phosphorous acid group, phenolic hydroxyl group or a salt of one of the groups, and a is 1, 2 or 3, with the proviso that, if the monomer of the formula contains a carboxyl group or a carboxylate group, either (1) this monomer contains at least one further radical A having a different one of the definitions specified for A, or (2) at least one additional monomer of the formula is also used in which A has a different one of the definitions specified for A; and (b) at least one other aliphatically unsaturated monomer.

The term polymer also includes macromer, which is a precursor to a polymer, and which can be incorporated into the lens of the invention.

It is believed that the polymers used in the invention are biospecific polymers, i.e. polymers capable of biospecific molecular recognition. It is known in the art that random attachment of functional groups to certain polymers results in biospecificity. It is also known that the level of biological activity varies with the composition of the copolymer, such that there may be a maximum in activity at some intermediate composition between maximum and zero content of the functional groups. In the present invention, it is preferable for the polymer to have random substitution of several substrates, e.g. carboxylate and sulfonate groups. A general background regarding biospecificity of random copolymers may be found in Jozefowicz and Jozefonvicz, Randomness and Biospecificity: random copolymers are capable of biospecific molecular recognition in living systems, Biomaterials, 18 (1997) 1633-1644 (incorporated herein by reference).

Monomers, which are suitable for preparing the polymers, include but are not limited to, acrylic acid, methacrylic acid, 4-vinylsalicylic acid, itaconic acid, vinylacetic acid, cinnamic acid, 4-vinylbenzoic acid, 2-vinylbenzoic acid, sorbic acid, caffeic acid, maleic acid, methylmaleic acid, dimethylmalcic acid, dihydroxymaleic acid, isocrotonic acid, fumaric acid, methylfumaric acid, allylacetic acid and the alkali metal salts, specially the sodium salts, of these acids, vinylsulfonic acid, allylsulfonic acid, methallylsulfonic acid, 4-styrenesulfonic acid, 2-styrenesulfonic acid, vinyltoluene-sulfonic acid, 2-acrylamido-2-methyl-1-propanesulfonic acid (AMPS), 4-carboxy styrenesulfonic acid and the alkali metal salts of these sulfonic acids, diprimary 1,3-butadiene-1,4-diol diphosphate, and the corresponding salts. Polymers and contact lenses are made from these monomers by conventional methods.

An embodiment of the present invention is a method for preventing microbial adhesion and growth comprising attaching a polymer to a surface of an ophthalmic device, wherein said polymer has carboxylate and sulfonate groups. The molar ratio of the carboxylate groups to sulfonate groups is preferably greater than about 2, more preferably about 2 to about 4.

Preferably, the polymer is attached to at least the surface of the ophthalmic device which contacts the cornea or the surface of the ophthalmic device which contacts the interior of the eyelid during conventional use of the ophthalmic device. More preferably, the polymer is attached to the surface of the ophthalmic device which contacts the cornea and the surface which contacts the interior of the eyelid during conventional use. Most preferably, the polymer is attached to all of the surfaces of the ophthalmic device.

The ophthalmic device with the attached polymer may be placed in a fluid, such as tears, storage fluid, such as the fluid used to store contact lenses during shipment, before use and between uses by the consumer or the fluid used to clean and/or disinfect the contact lenses between uses by the consumer. A synthetic tear fluid may be used to mimic these fluid examples in in vitro testing.

In one embodiment, the polymer is attached to the surface of an ophthalmic device, including but not limited to a contact lens, by methods known to those skilled in the art including but not limited to surface grafting, plasma polymerization, mold transfer coating or tampo printing. When the ophthalmic device is placed in contact with tear fluid or other bodily fluid, such as plasma, adhesive proteins, such as fibronectin, attach to one or more binding sites in or on the ophthalmic device. These sites may be on the surface of the device, which contacts the cornea, eyelid or all of those surfaces. The ophthalmic device is preferably a contact lens, preferably comprising poly(methyl)methacrylate polymer, silicon acrylate polymer, fluoroacrylate polymer, fluoroether polymer, polyacetylene polymer, polyimide polymer, hydrogels, silicone materials, acrylic materials, fluorocarbon materials, etafilcon A, genfilcon A, galyfilcon A, lenefilcon A, polymacon, acquafilcon A, balafilcon A, lotrafilcon A, lotrafilcon B, silicone hydrogels, or combinations thereof.

Without being limited to the mechanism, it is thought that when the binding of the adhesive protein occurs such that the binding sites are the same sites which would be used by microbes for binding to the surface of the ophthalmic device, the binding of the adhesive protein may result in substantially reducing microbial (including but not limited to bacterial) adhesion to the device, preferably about 90% reduction takes place as compared to a device without the polymer. The microbes may include but are not limited to Pseudomonas aeruginosa, Acanthamoeba species, Staphylococcus aureus, Escherichia coli, Staphylococcus epidermis, Serratia marcesens, or combinations thereof.

The polymer includes but is not limited to methyl methacrylate copolymers, methacrylic acid copolymers, sodium styrenesulfonate copolymers, methyl methacrylate-methacrylic acid-sodium styrene sulfonate random copolymers or combinations thereof. The molar ratio of carboxylate group to sulfonate group is about 2 to about 4, preferably greater than about 2.

### EXAMPLE 1

Methylmethacrylate (“MMA”) and methacrylic acid (“MA”) were distilled prior to use. Sodium styrene sulfonate (“SS”) was determined to contain 9.74% w/w H₂O, which was accounted for in the stoichiometric calculations.

MMA (6.0 ml, 56.1 mmol), MMA (1.27 ml, 15 mmol), and SS (0.854 g, 3.74 mmol) were charged into the reaction vessel, and DMSO (35 ml) was then added. A solution of AIBN (2,2′-Azobisisobutyronitrile, CAS # 78-67-1) in DMSO (dimethyl sulfoxide) (5.5 mg/ml) was prepared separately, and 1.0 ml of this was introduced into the reaction vessel. The set-up was purged of oxygen by using liquid N₂ freeze-pump-thaw method (repeated three times). The reaction was then heated to 75° C., and allowed to proceed for 16 to 18 hours under nitrogen. Diluting the reaction with 30 ml of methanol, and dropwise addition of the resulting solution to isopropanol (900 ml) precipitated the polymer. The polymer was filtered, and dried in vacuo to yield a white powder. Two grams of the polymer was further purified by stirring the polymer in 60-70 ml of deionized water at about 50-60 C, followed by filtration, and further washing (two times with 15 ml) with deionized water.

The water washed polymer after drying in vacuo was analyzed by ¹H NMR, which contained the expected peaks and showed no traces of residual monomers. The carboxylate/sulfonate ratio was determined to be 3.45 (the theoretical value was 4.0). The molecular weight (SEC, light scattering in DMF) was 187,000.

### EXAMPLE 2

MMA and MA were distilled prior to use. SS contained 9.96% w/w H₂O, which was accounted for in the stoichiometric calculations.

A 100 ml 3-neck round bottom flask equipped with a magnetic stir bar, a reflux condenser with nitrogen inlet, a glass stopper, and a rubber septum was purged under positive nitrogen flow for 30 minutes. MMA (18.0 ml, 168 mml), MA (3.81 ml, 45 mmol), SS (2.56 g, 11.2 mmol) and DMSO (45 ml) were blended together in a 120 ml screw cap amber jar. This monomer mixture was then transferred to the 100 ml 3-neck flask. A solution of AIBN in DMSO (16.5 mg in 3 ml of DMSO) was prepared separately and introduced into the reaction vessel. Nitrogen gas was bubbled through the reaction mixture for 30 minutes, while stirring, to deoxygenate the mixture. The reaction was then heated to 75 C, and allowed to proceed for 16 hours under nitrogen. Diluting the reaction with 250 ml methanol and dropwise addition of the resulting solution to isopropanol (2.1 L) precipitated the polymer. The supernatant was discarded, and the polymer was filtered and washed further with isopropanol (1×400 ml; 2×150 ml). The resulting material upon drying in vacuo at 65 C yielded 15.8 g (68.7% yield) of a white polymer. The polymer was pulverized in a commercial blender and 5.8 grams were further purified by stirring in 200 ml of deionized water at 60 C, followed by filtration and further washing with deionized water (2×50 ml), and drying in vacuo (5 mbar) at 65° C. for 6.5 hours to yield 5.2 g of a white powder.

The water-washed polymer was analyzed by ¹H NMR, which contained the expected peaks and showed no traces of residual monomers. The carboxylate/sulfonate ratio was determined to be 3.21 (theoretical value=4.0). The composition of the polymer was 75.6(MMA):18.6(MA):5.8(SS). Size exclusion chromatography (DMF against polystyrene standards) was used to determine a Mw=259,760; Mn=100,130.

### EXAMPLE 3

A polymer from Example 2 was dissolved into a 70:30 ethanol:ethyl lactate solvent solution to make 1.5% (w/v) solution of polymer. The solution was used to coat Zeonor 1060R (trademark) lens molds via spin coating according to the following procedure.

The 1.5% polymer from Example 2 coating solution was applied to the Zeonor front curve mold surface by dispensing approximately 3 μl of the solution into the center of a mold spinning at approximately 7500 rpm and thereafter spun for 8 seconds. During the last 2 seconds of spinning the excess coating near the edge of the mold was cleared using a pressurized air jet nozzle (˜15 psi). The back curve mold is coated similarly using the 1.5% polymer solution from Example 2 is used for the coating and the coating is applied to the mold spinning at 6000 rpm for 2 seconds followed by 6 seconds of spinning at 7500 rpm. Again, a pressurized air jet is used to remove excess coating near the edge of the mold for the last two seconds of spin time.

### EXAMPLE 4

MMA and MA were distilled prior to use. SS was determined to contain 9.74% w/w H₂O, which was accounted for in the stoichiometric calculations.

MMA (6.0 ml, 56.1 mmol), MA (1.27 ml, 15 mmol) and SS (0.854 g, 3.74 mmol) were changed into the reaction vessel, and DMSO (15 ml) was then added. A solution of AIBN in DMSO (5.5 mg/ml) was prepared separately and 1.0 ml was introduced into the reaction vessel. The reaction was deoxygenated by bubbling nitrogen through the monomer mixture for 30 minutes while stirring. The reaction was then heated to 75° C., and allowed to proceed for 16 to 18 hours under nitrogen. Diluting the reaction with methanol, and dropwise addition of the resulting solution to isopropanol (800 ml) precipitated the polymer. The polymer was filtered, and dried in vacuo at 65° C. The dried polymer was ground to a fine powder.

One gram of the polymer was soaked in 50 ml of isopropanol, filtered, then washed twice with 30 ml isopropanol and once with 50 ml of hexane and dried in vacuo at 65° C. to yield the “IPA-washed fraction.” One gram of the polymer was stirred into 50 ml of deionized water at 60 C, cooled to room temperature, filtered, and washed twice with 30 ml of deionized water and dried in vacuo at 65° C. to obtain the “water-washed fraction.”

The polymers in Table 1 below were made using the method described in Example 4, except that the molar ratios of the MMA, MA and SS were varied as indicated in the table.

TABLE 1

| | MMA | MA | SS | |
|---|---|---|---|---|
| Polymer | (mol %) | (mol %) | (Mol %) | MA/SS |
| Example 4 | 75 | 20 | 5 | 4 |
| (theoretical) | | | | |
| Example 4 | 78 | 16.3 | 5.7 | 2.86 |
| IPA-washed | | | | |
| fraction | | | | |
| (actual) | | | | |
| Example 4 | 76.9 | 17.6 | 5.5 | 3.2 |
| Water-washed | | | | |
| fraction | | | | |
| (actual) | | | | |
| Second Trial | | | | |
| Example 4A | 69 | 24.8 | 6.2 | 4 |
| (theoretical) | | | | |
| Example 4A | 66.1 | 25.3 | 8.6 | 2.94 |
| IPA-washed | | | | |
| fraction | | | | |
| (actual) | | | | |
| Example 4A | 73.9 | 19.2 | 6.9 | 2.78 |
| Water-washed | | | | |
| fraction | | | | |
| (actual) | | | | |
| Third Trial | | | | |
| Example 4B | 84 | 12.8 | 3.2 | 4 |
| (theoretical) | | | | |
| Example 4B | 84.6 | 11.5 | 3.9 | 2.95 |
| IPA-washed | | | | |
| fraction | | | | |
| (actual) | | | | |
| Example 4B | 83.2 | 12.7 | 4.1 | 3.1 |
| Water-washed | | | | |
| fraction | | | | |
| (actual) | | | | |

In Example 4A, the water-washed fraction had a significantly higher MMA content as compared to the IPA-washed fraction. Without being limited to mechanism, this suggests that water removed the more ionic chains in the sample. Washing the polymer with water did not change the MA/SS ratio significantly as compared to the EPA-washed fraction.

In Example 4 and 4B with the polymer with an intermediate to high overall MMA content, water washing did not significantly impact the composition or the MA/SS ratio of the polymer. Since the biospecific interactions of polymer chains are derived from the random functional group distribution, any purification technique that systemically alters the composition of the polymer (e.g. removal of highly ionic chains) will skew the distribution. It is preferable to preserve the original statistical distribution obtained during polymerization for MMA/MA/SS copolymers, and in order to do so, total (theoretical) ionic content of less than or equal to 25% on molar basis is preferred. The compositions in Table 1 labeled (actual) were determined from ¹H NMR spectra, as indicated in Example 5-14.

### EXAMPLES 5 TO 14

The polymers in Table 2 were made using the method described in Example 1, except that the molar ratios of the MMA, MA and SS were varied as indicated in the table.

TABLE 2

| | MMA | MA | SS | |
|---|---|---|---|---|
| | (mol %) | (mol %) | (mol %) | MA/SS |
| Example 5 | 65 | 30 | 5 | 6 |
| (theoretical) | | | | |
| Example 5 | 68.1 | 26 | 5.9 | 4.41 |
| (actual) | | | | |
| Example 6 | 70 | 30 | 0 | — |
| (theoretical) | | | | |
| Example 6 | 80.6 | 19.4 | 0 | — |
| (actual) | | | | |
| Example 7 | 67.5 | 22.5 | 10 | 2.25 |
| (theoretical) | | | | |
| Example 7 | 68.7 | 19.6 | 11.7 | 1.68 |
| (actual) | | | | |
| Example 8 | 75 | 15 | 10 | 1.5 |
| (theoretical) | | | | |
| Example 8 | 82.7 | 5.7 | 11.6 | 0.49 |
| (actual) | | | | |
| Example 9 | 60 | 30 | 10 | 3 |
| (theoretical) | | | | |
| Example 9 | 62.5 | 26.4 | 11.1 | 2.38 |
| (actual) | | | | |
| Example 10 | 75 | 15 | 10 | 1.5 |
| (theoretical) | | | | |
| Example 10 | 77.5 | 10.6 | 11.9 | 0.89 |
| (actual) | | | | |
| Example 11 | 60 | 30 | 10 | 3 |
| (theoretical) | | | | |
| Example 11 | 59.5 | 28.2 | 12.3 | 2.29 |
| (actual) | | | | |
| Example 12 | 70 | 25 | 5 | 5 |
| (theoretical) | | | | |
| Example 12 | 70.1 | 24.4 | 5.5 | 4.44 |
| (actual) | | | | |
| Example 13 | 75 | 20 | 5 | 4 |
| (theoretical) | | | | |
| Example 13 | 78.2 | 16.9 | 4.9 | 3.45 |
| (actual) | | | | |
| Example 14 | 75 | 25 | 0 | — |
| (theoretical) | | | | |
| Example 14 | 88 | 12 | 0 | — |
| (actual) | | | | |

The compositions in Table 2 labeled (actual) were determined from ¹H NMR spectra (270 MHz), acquired in deuterated DMSO. The peaks used to calculate the relative ratios were the aromatic protons of the SS residues (δ˜6.8-7.6 ppm), the methyl ester protons from MMA (δ˜3.5 ppm), and the combined peak (δ˜0.2-2.2 ppm) from the β-methyl protons of MMA/MA and the β-methylene protons of MMA/MA/SS.

The polymers were purified, prior to ¹H NMR analysis, by washing with deionized water. Two grams of the polymer was stirred into 60-70 ml of deionized water at 50 to 60° C., cooled to room temperature, filtered, and washed twice with 30 ml of deionized water and dried in vacuo at 65° C. The ¹H NMR spectra did not show any evidence of residual monomers.

### EXAMPLE 15

Bacteriophobic and bacteriostatic properties in the presence of blood plasma and/or synthetic tear fluid containing fibronectin or other adhesive protein are dependent on the sulfonate and carboxylate compositions of the random tercopolymers. For the ratio of COO⁻/SO₃⁻ ranging between 1 and 1.4, the polymers exhibit bacteriophobic effect and eukaryotic cells proliferation inhibition. For the ratio ranging between 0 to 0.5 and 3 to 4, there are bacteriophobic properties but almost normal eukaryotic cell proliferation. For the ratio above 4, there is normal eukaryotic cell proliferation and bacterial adhesion.

Silicone hydrogels were coated using the method in Example 2 with random biospecific polymers (P,Q,R,S) with a ratio of COO—/SO3- ranging between 0 and 5.

TABLE 3

| | | Composition | |
|---|---|---|---|
| | Theoretical | determined by | |
| | composition | NMR | Ratio = |
| | MMA/MA/SS | MMA/MA/SS | MA/SS |
| P | 60/30/10 | 59.5/28.2/12.3 | 2.29 |
| Q | 75/20/5 | 78.2/16.9/4.9 | 3.45 |
| R | 70/25/5 | 70.1/24.4/5.5 | 4.44 |
| S | 70/0/30 | 53/0/47 | 0 |
| T | Poly HEMA coated | — | — |
| | control | | |
| U | Uncoated silicone | — | — |
| | hydrogel | | |

Overnight cultures of Pseudomonas aeruginosa and Staphylococcus aureus were prepared separately by the incubation of colonies selected from the agar plate in 1 ml of broth at 30° C. or 37° C., respectively. The 1 ml of saturated bacterial suspensions were harvested by centrifugation at 3500 rpm for 10 minutes. The supernatant was discarded and 1 ml of fresh broth was added to the pellet. The solutions were vortex-mixed to ensure that bacteria were in suspension.

Columbia agar and Brain-Heart infusion were used for Pseudomonas aeruginosa (“P. aeruginosa”) cultures, which were performed at 30° C. Mueller-Hinton agar and Mueller-Hinton broth were used for Staphylococcus aureus (“S. aureus”) cultures, which were performed at 37° C.

50 μl of tritiated thymidine (1 mci/ml) was added to broth containing about 107 cfu/ml of the bacteria. The suspensions were incubated for 4 hours at 30° C. in the case of P. aeruginosa or 3 hours at 37° C. in the case of S. aureus to obtain exponential cultures. After the incubation periods, the bacterial suspensions were harvested twice at 3500 rpm for 10 minutes to remove the excess unbound radioactivity. PBS with Ca++ and Mg++ was finally added to the bacterial pellet to obtain suitable bacterial dilutions (10⁶-10⁷ cfu/ml) determined from the standard curve corresponding to the absorbency versus cfu and the suspensions were mixed with a vortex mixer.

Bacterial concentrations were controlled by measuring viable cells by cfu counting. Bacterial solutions were diluted in order to obtain 30 to 300 colonies spread on the agar plates.

100 μl of each bacterial suspension was added to 2 ml of scintillation fluid in the vials and the radioactivity of bacterial dilutions was measured on a β-automatic liquid scintillation counter.

Procedure for Mediated Bacterial Adhesion Assay

The lenses were aseptically transferred into cell culture boxes and washed five times in 2 ml of Phosphate-buffered saline (“PBS”). The lenses were then incubated for 1 hour at room temperature under stirring with synthetic tear fluid. The synthetic tear fluid is human plasma diluted at 5% in PBS supplemented with Lysozyme (4.7 g/l) and lactoferrin (1.7 g/l). After the incubation periods, lenses were washed three times with PBS and were then transferred to new cell culture boxes. The bacterial adhesion is reported as % adhesion of the bacteria in suspension which are adhered to the lens.

Synthetic tear fluid with coated lenses and uncoated lenses were incubated with 1 ml of two concentrations of the radio-labeled bacteria for 1 hour at 30° C. or at 37° C. (respectively for the bacteria strains) under stirring. After five washings of the lenses with buffer, the lenses were transferred to counting vials, 10 ml of scintillation fluid was added, the solutions were mixed with a vortex mixer and the radioactivity incorporated by the adhered bacteria was measured with a β-counter. The bacterial adhesion is reported as % adhesion of the bacteria in suspension which are adhered to the lens.

Procedure for Non-Mediated Bacterial Adhesion Assay

Lenses were aseptically transferred into cell culture boxes and washed five times in 2 ml of phosphate-buffered saline (“PBS”). The lenses were then incubated with 1 ml of two concentrations of the radio-labeled bacteria for 1 hour at 30° C. or at 37° C. (respectively for the bacteria strains) under stirring. After five washings of the lenses with PBS, the lenses were transferred to counting vials, 10 ml of scintillation fluid was added, the solutions were mixed with a vortex mixer and the radioactivity incorporated by the adhered bacteria was measured with a B-counter. The bacterial adhesion is reported as the adhesion percentages of the bacteria in suspension, which are adhered to the lens.

Procedure for Bacterial Proliferation Assay

Synthetic tear fluid coated lenses were first incubated for 1 hour with suspended bacteria. The unbound bacteria were removed by washing the lenses 5 times in buffer. The lenses were then transferred to new tubings containing 1 ml of the selected media. They were then incubated at 37° C. under stirring for various periods of time ranging between 0 and 5 hours. At the end of the incubation, both the number of surface-bound organisms and the number bacteria in suspension (Br) were determined.

The number of surface-bound bacteria (Bs) was determined by detaching the organisms from the surface according to the following scheme. Washing the lenses in PBS, and incubating the lenses in 1 ml of a solution of trypsin for 5 min. at 37° C. under smooth stirring. Then, the solutions is vortex mixed and sonicated for 3 minutes, followed by 3 washings of the lenses in PBS. The pool of the washings are spun at 3500 rpm for 15 minutes. The bacterial pellet is resuspended in PBS and counted for cfu after suitable dilutions on agar gel.

The efficiency of the detachment of the Bs has been controlled by the comparison of the number of bacteria adhered to the lenses and measured before and after detachment by an agar overlay method in common use by those of ordinary skill in the art. This showed that in the case of P. aeruginosa about 80% of the Bs are detached and alive. Whereas, 90% of the Bs are detached and alive for S. aureus.

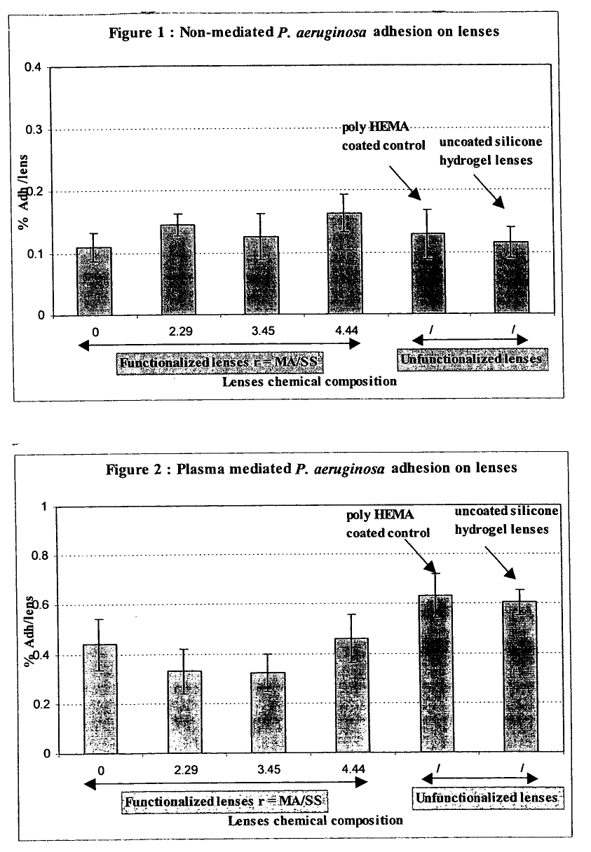

As seen in FIG. 1, non-mediated P. aeruginosa adhesion is measured on control lenses (unfunctionalized lenses) and lenses coated with the tercopolymers (functionalized lenses); in this case, the lenses were characterized by the MA/SS ratio. The non-mediated adhesion percentages were not statistically different for any of the lenses under consideration.

As seen in FIG. 2, plasma mediated P. aeruginosa adhesion is measured on control lenses (unfunctionalized lenses) and lenses coated with the tercopolymers (functionalized lenses); in this case, the lenses were characterized by the MA/SS ratio. The mediated bacterial adhesion is from 4 to 6 time greater than the non-mediated adhesion.

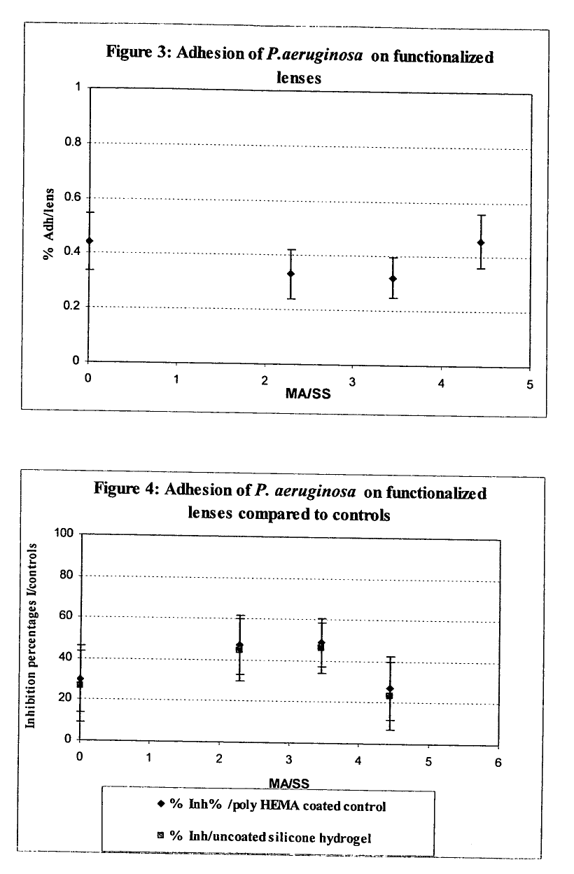

Lenses coated with the acrylic copolymers have minimal adhesion percentages for MA/SS ratios of 2.29 and 3.45 as seen in FIG. 3. The inhibition of the mediated bacterial adhesion is evidence by the comparison between bacterial adhesion random biospecific lenses (functionalized lenses) and controls (poly HEMA coated control and uncoated silicone hydrogel) and is calculated by the ration:

I=[% adh/lens (control)−% adh/lens (random biospecific lenses)×100]/[% adh/lens (random biospecific lenses)]

This ratio evidences the inhibitory or promoting effect of the bacterial adhesion on random biospecific lenses as compared to controls.

FIG. 4 shows the variation of the I ratio as a function of MA/SS ratio in the copolymers. Inhibition of the bacterial adhesion reached as high as 50% for MA/SS ratios of 2.29 and 3.45. When MA/SS ratio is 0 (and the copolymer has only MMA and SS monomers), the inhibition of the bacterial adhesion is significantly lower (20%) as compared to 50%.

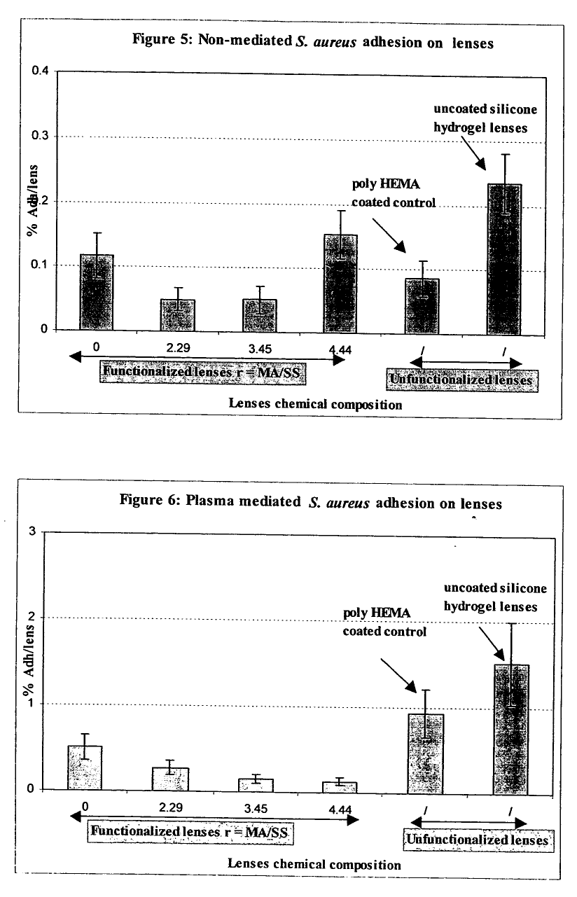

Non-mediated bacterial adhesion for S. aureus on polyHEMA coated control lenses (poly-Hema lenses made as is known in the art) is significantly lower than on uncoated silicone hydrogel lenses as is shown in FIG. 5. The non-mediated adhesion values on copolymers with MA/SS ratio of 0 and on MA/SS ratio of 4.44 are between the adhesion values for the controls. On copolymers with MA/SS ratios of 2.29 and 3.45, the adhesion values are significantly lower compared to other copolymers and controls.

The mediated bacterial adhesion is from 5 to 10 times higher on the nonfunctionalized lenses and from 2 to 5 times higher on functionalized lenses compared to the non-mediated bacterial adhesion as seen in FIG. 6. Adhesion of S. aureus on poly HEMA coated control lenses is 0.93+−0.28%/lens and 1.5+−0.48%/lens on nonfunctionalized lenses. Functionalized lenses with either MMA and SS copolymers (MA/SS=0) have bacterial adhesions of 0.5+−0.14%/lens, which is significantly lower than nonfunctionalized. Functionalized lens with (MA, SS, MMA), the mediated adhesion of S. aureus is significantly lower than that observed in the nonfunctionalized. MA, SS and MMA are all present in the tercopolymer.

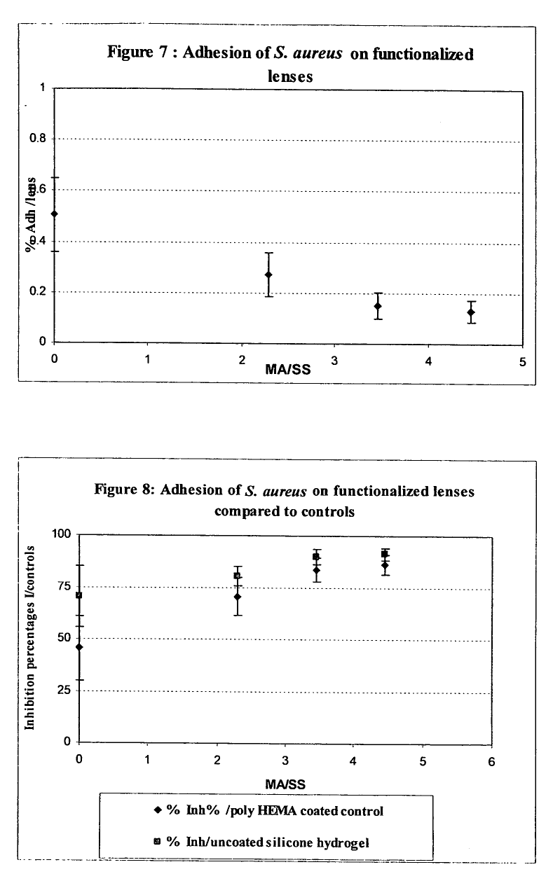

FIG. 7 shows that functionalized lenses have low adhesion values for MA/SS ratio equal to 2.29, 3.45 and 4.44. The inhibition of the mediated bacterial adhesion is evidence by the comparison between bacterial adhesion on functionalized lenses and nonfunctionalized lenses and is calculated by the I formula described above.

The variation of inhibition (I) as a function of MA/SS ratio in the copolymers is shown in FIG. 8. The inhibition reaches a maximum of 80% for MA/SS equal to 3.45 and 4.44. For MA/SS=0 (the copolymer has only MMA and SS monomers), the inhibition of bacterial adhesion is significantly lower compared to the increased MA/SS ratio.

Therefore, lenses coated with MMA, MA and SS copolymers show bacteriophobic properties when the lenses are exposed to synthetic tear fluid with regard to the mediated adhesion of P. aeruginosa and S. aureus. The maximum of the bacteriophobic effect for both strains is observed for the lenses coated with the tercopolymer MA/SS 2.29 and 3.45. A tercopolymer means a copolymer created with at least three monomers. It is noticeable that MMA, SS copolymer (MA/SS=0) coated lenses are less bacteriophobic in the same conditions.

Without being held to a mechanism, the mediated bacterial adhesion depends on the chemical composition of the lenses and the surface of the lenses. Adhesion of P. aeruginosa on controls lenses is 0.62+−0.02%/lens and for lenses coated with either MMA and SS copolymer (MA/SS=0) the bacterial adhesion is 0.44+−0.1%/lens which might be lower than that observed on controls. The standard deviations are a little too large to conclude.

For lenses coated with the terpolymers (MA, SS, MMA), the mediated adhesion of P. aeruginosa is significantly lower than that observed in the case of the controls especially for the ratio 2.29 and 4.44.

Bacteriophobic Effect

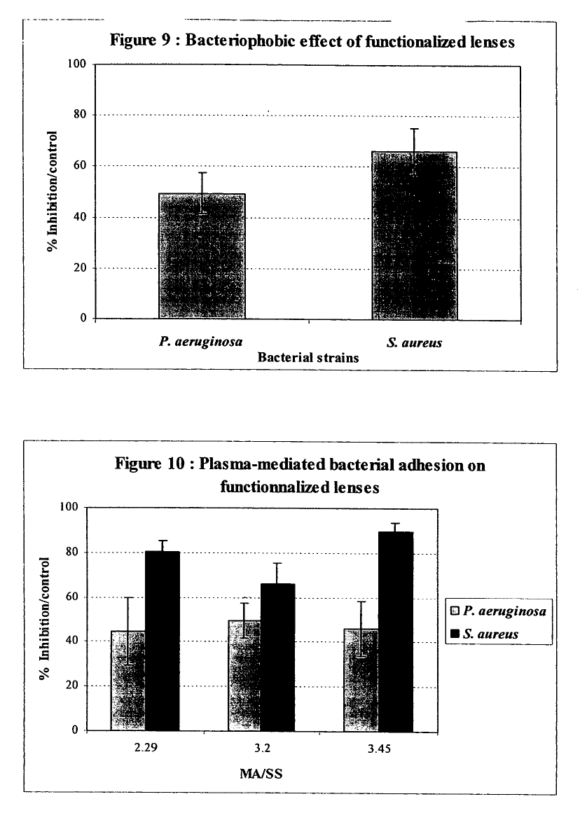

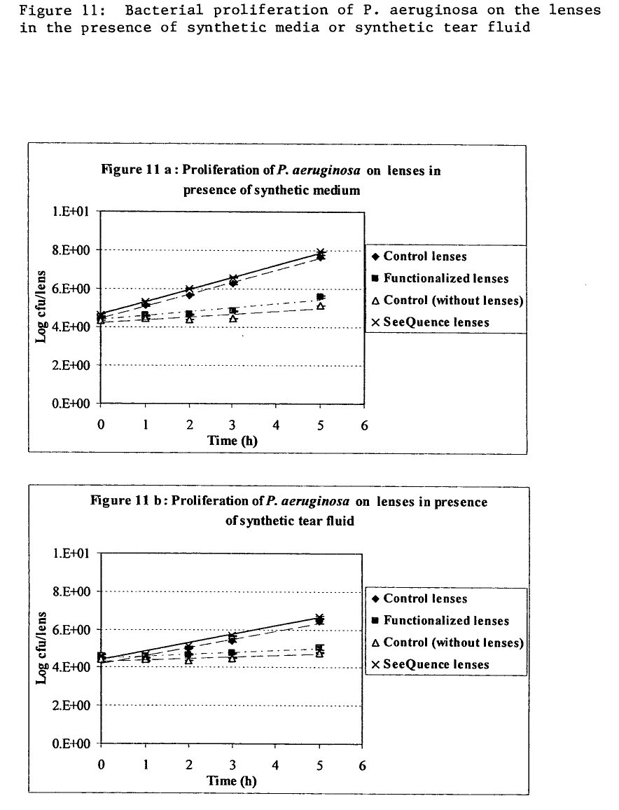

In FIG. 9, the inhibition of the bacterial adhesion reaches 50% in the case of P. aeruginosa and 70% in the case of S. aureus. There was no significant difference in abilities of the lenses to inhibit bacterial adhesion for various MA/SS ratios as seen in FIG. 10. Therefore, it is expected that the bacteriophobic properties of the copolymer will not be dramatically affected if the copolymerization process leads to small variations of the MA/SS ratio.

### EXAMPLE 16

Bacterial proliferation studies were conducted on functionalized lenses with a surface coating described in this patent, control silicon hydrogel lenses with a poly HEMA coating, SeeQuence brand contact lenses and a control fluid with no lenses. Lenses were contacted with the tear-like fluid for one hour. The lenses were washed three times with phosphate buffered saline. The lenses were then incubated with about 10⁸ bacteria/ml for one hour. The lenses were rinsed and placed in a synthetic growth medium. The number of bacteria were measured at 1, 2, 3 and 5 hours.

The functionalized lenses have a long time lag (>2 hours) which is then followed by an exponential proliferation. The number of bacteria adhered to the functionalized lenses is about 35 times lower (for P. aeruginosa) and 8 times lower (for S. aureus) as compared to the control lenses.

TABLE 4
Proliferation parameters

| | | | | | |
|---|---|---|---|---|---|
| | Δlog | | | | |
| Lenses | (Log | k | | | |
| types | cfu/lens) | (generations/h) | g (min) | S | R |

Pseudomonas aeruginosa proliferation in presence of synthetic
medium

| | | | | | |
|---|---|---|---|---|---|
| Control | 3.2 ± 0.02 | 2.1 ± 0.12 | 28.6 ± 1.7 | 184 ± 11 | 251 ± 11 |
| lenses | | | | | |
| Functionalized | 1.05 ± 0.1 | 1.25 ± 0.07 | 47.8 ± 2.8 | 110 ± 6.6 | 1.8 ± 0.45 |
| Lenses | | | | | |
| SeeQuence | 3.3 ± 0.1 | 2.2 ± 0.09 | 27.3 ± 1.13 | 193 ± 8 | 340 ± 33 |
| Lenses | | | | | |
| Control | 0.8 ± 0.05 | 1.14 ± 0.07 | 52.7 ± 3.3 | 100 ± 00 | 1 ± 00 |
| (without | | | | | |
| lenses) | | | | | |

Staphylococcus aureus proliferation in presence of synthetic
medium

| | | | | | |
|---|---|---|---|---|---|
| Control | 3.2 ± 0.13 | 2.11 ± 0.17 | 28.4 ± 2.3 | 144 ± 12 | 57 ± 16 |
| lenses | | | | | |
| Functionalized | 1.4 ± 0.01 | 1.5 ± 0.16 | 40 ± 4 | 103 ± 10 | 1.03 ± 0.02 |
| Lenses | | | | | |
| SeeQuence | 3.3 ± 0.1 | 2.24 ± 0.01 | 26.73 ± 0.2 | 1152 ± 1 | 77 ± 19 |
| Lenses | | | | | |
| Control | 1.43 ± 0.02 | 1.47 ± 0.12 | 40.8 ± 3.4 | 100 ± 00 | 1 ± 00 |
| (without | | | | | |
| lenses) | | | | | |

TABLE 5
Proliferation parameters

| | | | | | |
|---|---|---|---|---|---|
| | Δlog | | | | |
| Lenses | (Log | k | | | |
| types | cfu/lens) | (generations/h) | g (min) | S | R |

Pseudomonas aeruginosa proliferation in presence of synthetic tear
fluid

| | | | | | |
|---|---|---|---|---|---|
| Control | 2 ± 0.25 | 1.66 ± 0.003 | 36.1 ± 0.06 | 346 ± 0.6 | 45.6 ± 25 |
| lenses | | | | | |
| Functionalized | 0.5 ± 0.06 | 0.53 ± 0.16 | 117 ± 35 | 111 ± 33 | 1.3 ± 0.2 |
| Lenses | | | | | |
| SeeQuence | 2.0 ± 0.09 | 1.7 ± 0.03 | 35.6 ± 0.6 | 351 ± 6 | 50 ± 10 |
| Lenses | | | | | |
| Control | 0.38 ± 0.03 | 0.5 ± 0.017 | 124.3 ± 4.5 | 100 ± 00 | 1 ± 00 |
| (without | | | | | |
| lenses) | | | | | |

Staphylococcus aureus proliferation in presence of tear-like fluid
Proliferation parameters

| | | | | | |
|---|---|---|---|---|---|
| Control | 1.5 ± 0.08 | 1.24 ± 0.05 | 48 ± 2 | 175 ± 7 | 8.6 ± 1.6 |
| lenses | | | | | |
| Functionalized | 0.6 ± 0.02 | 0.6 ± 0.07 | 102.8 ± 13 | 82.7 ± 10 | 1.05 ± 0.04 |
| Lenses | | | | | |
| SeeQuence | 1.65 ± 0.2 | 1.34 ± 0.1 | 45 ± 3.4 | 188 ± 14 | 12.65 ± 5 |
| Lenses | | | | | |
| Control | 0.56 ± 0.09 | 0.7 ± 0.13 | 85.12 ± 15 | 100 ± 00 | 1 ± 00 |
| (without | | | | | |
| lenses) | | | | | |

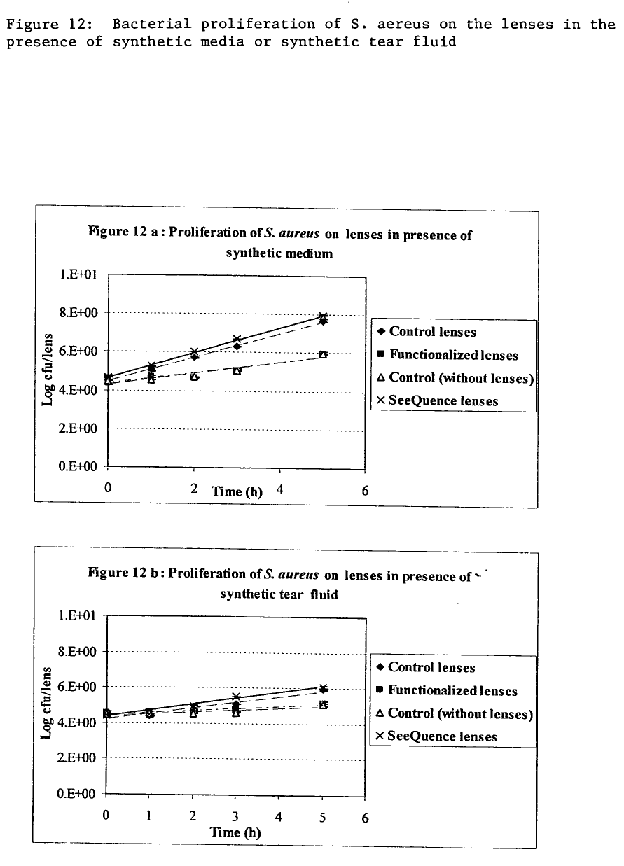

As seen in FIGS. 11 and 12, adhered bacterial proliferation is exponential. However, proliferation in suspension is almost non-existent in the presence of synthetic-tear fluid and a longer time lag followed by exponential proliferation is seen in the synthetic media.

In order to compare the rates of the proliferation of the bacteria onto the lenses and the proliferation in suspension in absence of any lenses the following characteristics were taken into account:

The increase of the bacterial population (ΔLog) after 5 hours of proliferation, expressed in Log cfu/mL and calculated from the following relation:

Δ log=Log N5−Log N0(log cfu/lens or Log cfu/mL in the case of control) with N0: bacterial concentration introduced initially (with 0 Hours of the proliferation)

N5: bacterial concentration obtained after 5 hours of proliferation.

The average constant rate of proliferation (k) expressed in a number of generations per hour and calculated from the following relation

$$k = \frac{Log Nn1 - Log Nn2}{0.30 \times t} ( generations \text{/} hour )$$

with, Nn1: the number of the bacteria at the moment t1

Nn2: the number of the bacteria at the moment t2

T: the time necessary to increase the population number from Nn1 to Nn2

The average generation time or double time (g) expressed in hour and represented by the reverse of the average constant rate of growth (k) g−1/k (hour per generation)

The stimulation of the proliferation rates (S) of the bacteria onto the lenses as compared to control (without lenses), calculated from the following relation:

$$S = \frac{k - lenses \times 100}{k ( control )}$$

The stimulation of the proliferation (R) as the number of generated bacteria after 5 hours as compared to control, calculated from the following relation:

$$R = \frac{N5 lenses / N0 lenses}{N5 control / N0 control}$$

with, N0 lenses: the number of the total proliferation on lenses after 0 hours of the incubation time

N5 lenses: the number of the total proliferation on lenses after 5 hours of the incubation time

N0 control: the number of the total proliferation in suspension after 0 hours of the incubation time

N5 control: the number of the total proliferation in suspension after 5 hours of the incubation time

Lenses incubated in the presence of synthetic tear fluid as a proliferation medium show that after 5 hours, the number of bacteria generated by the proliferation of the adhered bacteria as compared to the control in suspension is about 40 times in P. aeruginosa and 10 times in S. aureus.

It is apparent from this example that the functionalized lenses have approximately the same microbial growth as seen in the ocular environment without contact lenses and have a decreased microbial growth compared to that seen with contact lenses in having adhered microbes, including, but not limited to bacteria.

It is to be understood that while the invention has been described in conjunction with the detailed description thereof, that the foregoing description is intended to illustrate and not limit the scope of the invention, which is defined by the scope of the appended claims. Other aspects, advantages, and modifications are evident from a review of the following claims.

What is claimed is:

1. A method of preventing microbial attachment and growth comprising
Attaching a polymer to a surface of an ophthalmic device, wherein said polymer has a molar ratio of carboxylate groups to sulfonate groups of greater than about 2.

2. The method of claim 1 wherein said surface comprises one or more surfaces in contact with tears and placed adjacent to a cornea during conventional use of said device.

3. The method of claim 1 wherein said surface comprises one or more surfaces in contact with tears and placed adjacent to an interior of an eyelid.

4. The method of claim 1 wherein said ophthalmic device comprises a contact lens.

5. The method of claim 4 wherein said contact lens comprises one or more of the group consisting of poly(methyl)methacrylate polymer, silicon acrylate polymer, fluoroacrylate polymer, fluoroether polymer, polyacetylene polymer, polyimide polymer, hydrogels, silicone materials, acrylic materials, fluorocarbon materials, copolymers of any of the foregoing, etafilcon A, genfilcon A, galyfilcon A, lenefilcon A, polymacon, acquafilcon A, balafilcon A, lotrafilcon A, lotrafilcon B and silicone hydrogels.

6. The method of claim 1 further comprising placing the ophthalmic device in an ocular environment.

7. The method of claim 1 wherein said attaching occurs at one or more binding sites.

8. The method of claim 1 wherein said polymer comprises methyl methacrylate copolymers.

9. The method of claim 1 wherein said polymer comprises methacrylic acid copolymers.

10. The method of claim 1 wherein said polymer comprises sodium styrenesulfonate copolymers.

11. The method of claim 1 wherein said polymer comprises methyl methacrylate-methacrylic acid-sodium styrene sulfonate random copolymers.

12. The method of claim 1 wherein said molar ratio is about 2 to about 4.

13. The method of claim 1 further comprising substantially reducing microbial adhesion to said ophthalmic device as compared to said ophthalmic device without said polymer.

14. The method of claim 13 wherein said reducing is about 50% of microbes adhered to an ophthalmic device without said polymer.

15. The method of claim 13 wherein said reducing is about 90% of microbes adhered to an ophthalmic device without said polymer.

16. The method of claim 13 wherein said microbe comprises one or more of the group consisting of Pseudomonas aeruginosa, Acanthamoeba species, Staphylococcus aureus, Escherichia coli, Staphylococcus epidermis, and Serratia marcesens.

17. A method of preventing microbial attachment and growth comprising
placing an ophthalmic device on a cornea, said ophthalmic device comprising one or more surfaces, wherein a polymer is attached to said one or more surfaces, said polymer has a molar ratio of carboxylate group to sulfonate group of greater than about 2.

18. A method of promoting ocular health comprising reducing microbes adhered to an ophthalmic device, wherein said reducing comprises endowing a surface of an ophthalmic device with a random biospecific polymer that reduces microbial adhesion to the device.

19. A method of promoting ocular health comprising reducing microbes adhered to an ophthalmic device, wherein said reducing comprises endowing a surface of an ophthalmic device with a random biospecific polymer that reduces microbial growth on the device.

20. A method of promoting ocular health comprising reducing microbes adhered to an ophthalmic device, wherein said reducing comprises
attaching a polymer to a surface of an ophthalmic device, wherein said polymer has a molar ratio of carboxylate groups to sulfonate groups of greater than about 2.

21. The method of claim 20 wherein said surface comprises one or more surfaces in contact with tears and placed adjacent to a cornea during conventional use of said device.

22. The method of claim 20 wherein said surface comprises one or more surfaces in contact with tears and placed adjacent to an interior of an eyelid.

23. The method of claim 20 wherein said ophthalmic device comprises a contact lens.

24. The method of claim 23 wherein said contact lens comprises one or more of the group consisting of poly(methyl)methacrylate polymer, silicon acrylate polymer, fluoroacrylate polymer, fluoroether polymer, polyacetylene polymer, polyimide polymer, hydrogels, silicone materials, acrylic materials, fluorocarbon materials, etafilcon A, genfilcon A, galyfilcon A, lenefilcon A, polymacon, acquafilcon A, balafilcon A, lotrafilcon A, lotrafilcon B and silicone hydrogels.

25. The method of claim 20 wherein said attaching occurs at one or more binding sites.

26. The method of claim 20 wherein said polymer comprises methyl methacrylate copolymers.

27. The method of claim 20 wherein said polymer comprises methacrylic acid copolymers.

28. The method of claim 20 wherein said polymer comprises sodium styrenesulfonate copolymers.

29. The method of claim 20 wherein said polymer comprises methyl methacrylate-methacrylic acid-sodium styrene sulfonate random copolymers.

30. The method of claim 20 wherein said molar ratio is about 2 to about 4.

31. The method of claim 20 further comprising substantially reducing microbial adhesion to said ophthalmic device as compared to said ophthalmic device without said polymer.

32. The method of claim 31 wherein said reducing is about 50% of microbes adhered to an ophthalmic device without said polymer.

33. The method of claim 31 wherein said reducing is about 90% of microbes adhered to an ophthalmic device without said polymer.

34. The method of claim 31 wherein said microbe comprises one or more of the group consisting of Pseudomonas aeruginosa, Acanthamoeba species, Staphylococcus aureus, Escherichia coli, Staphylococcus epidermis, and Serratia marcesens.
